SEQUENCE LISTING (6 sequences)

SEQ ID NO: 1 - protein, 11 aa, Artificial sequence
  HIV-1 transactivator (Tat) Protein (47-57)
YGRKKRRQRRR

SEQ ID NO: 2 - RNA, 22 nt, Artificial sequence
  Passenger chain oligonucleotide
gcaacaccagucgaugggcugu

SEQ ID NO: 3 - RNA, 22 nt, Artificial seuqence
  hsa-miR-21-5p
uagcuuaucagacugauguuga

SEQ ID NO: 4 - protein, 9 aa, Artificial sequence
  Passenger chain oligonucleotide
RKKRRQRRR

SEQ ID NO: 5 - protein, 9 aa, Artificial sequence
  Passenger chain oligonucleotide
KKKKKKKKK

SEQ ID NO: 6 - protein, 9 aa, Artificial sequence
  Passenger chain oligonucleotide
RRRRRRRRR

(19) United States
(12) Patent Application Publication
(10) Pub. No.: US 2023/0212570 A1
(43) Pub. Date: Jul. 6, 2023

(54) CELL-PENETRATING PEPTIDE-MICRORNA CONJUGATES FOR INTRACELLULAR CELL DELIVERY

(71) Applicant: NORTH CAROLINA STATE UNIVERSITY, Raleigh, NC (US)

(72) Inventors: Ke Cheng, Raleigh, NC (US); Dashuai Zhu, Raleigh, NC (US)

(21) Appl. No.: 17/996,411

(22) PCT Filed: Apr. 20, 2021
(86) PCT No.: PCT/US2021/028080

§ 371 Date: Oct. 17, 2022

Related U.S. Application Data
(60) Provisional application No. 63/014,434, filed on Apr. 23, 2020.

(51) Int. Cl. C12N 15/113; A61K 47/64; A61K 47/54; C07K 14/005; A61K 31/7088

(52) U.S. Cl. CPC C12N 15/113; A61K 47/64; A61K 47/549; C07K 14/005; A61K 31/7088; C12N 2310/141; C12N 2310/3513; C12N 2740/16311; C12N 2740/16371

(57) ABSTRACT

Provided are compositions and methods useful in regenerating damaged tissue, especially cardiac tissue, by delivering to the site of injury an miRNA that can reduce, for example, the expression of phosphatase and tensin homolog (PTEN). The compositions and methods of the disclosure may be generally applied to deliver an miRNA to a cell or tissue such as, but not limited to, a neuron, a smooth muscle cell, or a tumor cell. The compositions comprise a transmembrane carrier peptide conjugated, optionally by a linker, to an oligonucleotide complementary to an miRNA. The carrier peptide facilitates the entry of the miRNA into cells and for delivery to a tissue of an animal or human may be mixed with an extracellular matrix-derived hydrogel carrier.

### CROSS-REFERENCE TO RELATED APPLICATIONS

This application claims priority to U.S. Provisional Patent Application Ser. No. 63/014,434 entitled “CELL-PENETRATING PEPTIDE-MICRORNA CONJUGATES FOR INTRACELLULAR CELL DELIVERY” filed on Apr. 23, 2020, the entirety of which is hereby incorporated by reference.

### SEQUENCE LISTING

This application contains a sequence listing filed in electronic form as an ASCII.txt file entitled “2214042420_ST25” created on Apr. 19, 2021. The content of the sequence listing is incorporated herein in its entirety.

### TECHNICAL FIELD

The present disclosure is generally related to compositions comprising a microRNA conjugated to a cell-penetrating peptide. The present disclosure is further generally related to methods of using a construct comprising a microRNA conjugated to a cell-penetrating peptide to induce repair of damaged tissue.

### BACKGROUND

Cell-based therapy holds promise for the regeneration of injured heart muscle (Luo et al., (2017) Circ. Res. 120: 1768-1775; Tang. et al., (2018) Nat. Biomed. Engineer. 2: 17-26; Tang et al., (2017) ACS Nano 11: 9738-9749; Tang et al., (2017) Nat. Commun. 8: 13724), have demonstrated that (Kreke et al., (2012) Expert Rev. Cardiovasc. Therapy 10: 1185-1194; Makkar et al., (2012) Lancet 379: 895-904; Chugh et al., (2012) Circulation 126: S54-S64; Rhee et al., (2018) Am Heart Assoc.; Qi et al., (2008) Chin. Med. J. Beijing 121: 544; Bellamy et al., (2015) J. Heart Lung Transplant 34: 1198-1207; Kim et al., (2014) J. Am. Coll. Cardiol. 64: 1681-1694; Joladarashi et al., (2015) J. Am. Coll. Cardiol. 66: 2214-2226). However, cell-based products must be carefully preserved to maintain their viability and activity until transplantation and there are also some risks involved in cell transplantation. The modes of action for cell therapy products remain elusive, making it difficult to standardize each cell lot. Recent meta-analyses indicate that cardiac cell therapies are overwhelmingly safe but only show none-to-marginal efficacy (Tang et al., (2018) Stem Cells Transl. Med. 7: 354-359). The development of cell-free and non-living therapeutics (e.g. proteins, RNAs) has the potential to revolutionize cardiovascular regenerative medicine. These therapeutics have compounded the evidence showing that the benefits of stem cell therapies mainly come from paracrine mechanisms instead of the direct differentiation of injected stem cells into cardiomyocytes (Chimenti et al., (2010) Circ. Res. 106: 971-980; Malliaras et al., (2011) Circulation 111.042598). The injected cells secrete proteins and nucleic acids to promote endogenous repair (Raposo and Stoorvogel (2013) J. Cell Biol. 200: 373-383).

### SUMMARY

One aspect of the disclosure, therefore, encompasses embodiments of a composition that can comprise a passenger chain oligonucleotide connected to a cell-penetrating peptide.

In some embodiments of this aspect of the disclosure, the cell-penetrating peptide and the passenger chain oligonucleotide can be connected by a linker.

In some embodiments of this aspect of the disclosure, the linker can be covalently conjugated to the cell-penetrating peptide and to the passenger chain oligonucleotide

In some embodiments of this aspect of the disclosure, the composition can further comprise an miRNA oligonucleotide having a nucleotide sequence selectively hybridizable to the passenger chain nucleotide sequence.

In some embodiments of this aspect of the disclosure, the passenger chain oligonucleotide can have an amino group at the 3′ terminus thereof.

In some embodiments of this aspect of the disclosure, the cell-penetrating peptide can be HIV-1 transactivator (Tat) Protein (47-57) having the amino acid sequence SEQ ID NO: 1.

In some embodiments of this aspect of the disclosure, the HIV-1 transactivator (Tat) Protein (47-57) can further comprise a cysteine residue conjugated to the amino terminus of the HIV-1 transactivator (Tat) Protein (47-57).

In some embodiments of this aspect of the disclosure, the linker can be (succinimidyl 4-(N-maleimidomethyl)cyclohexane-1-carboxylate) (SMCC).

In some embodiments of this aspect of the disclosure, the passenger chain oligonucleotide can have the nucleotide sequence of SEQ ID NO: 2.

In some embodiments of this aspect of the disclosure, the HIV-1 transactivator (Tat) Protein (47-57) can further comprise a cysteine residue conjugated to the amino terminus of the peptide SEQ ID NO: 1, and wherein the cysteine residue can be further conjugated to a linker, wherein the linker can be (succinimidyl 4-(N-maleimidomethyl)cyclohexane-1-carboxylate) (SMCC), and wherein the linker can be further conjugated to the passenger chain oligonucleotide, the passenger chain oligonucleotide having an amino group at the 3′ terminus thereof.

In some embodiments of this aspect of the disclosure, the composition has the formula I:

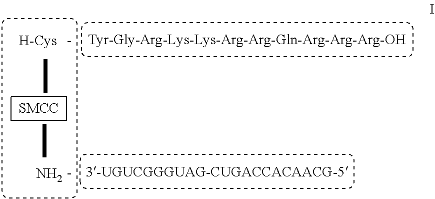

In some embodiments of this aspect of the disclosure, the miRNA can be miRNA-21 and can have a nucleotide sequence of SEQ ID NO: 3.

In some embodiments of this aspect of the disclosure, the composition can have the formula II:

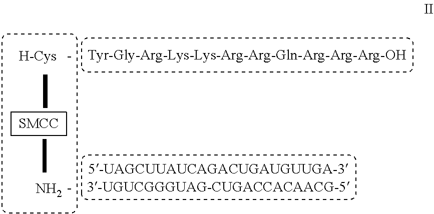

Another aspect of the disclosure encompasses embodiments of a method of delivering an miRNA to a cell, the method comprising contacting a cell with a composition comprising a passenger chain oligonucleotide connected to a cell-penetrating peptide, wherein the cell-penetrating peptide and the passenger chain oligonucleotide can be connected by a linker covalently conjugated to the cell-penetrating peptide and to the passenger chain oligonucleotide, and wherein the composition can further comprise an miRNA oligonucleotide having a nucleotide sequence selectively hybridized to the passenger chain nucleotide sequence.

In some embodiments of this aspect of the disclosure, the cell can be a cardiac cell.

In some embodiments of this aspect of the disclosure, the cardiac cell can be a cardiomyocyte.

In some embodiments of this aspect of the disclosure, the composition can be administered to a human or animal subject, and wherein the composition is admixed with a pharmaceutically acceptable carrier.

In some embodiments of this aspect of the disclosure, the pharmaceutically acceptable carrier can be a hydrogel generated from decellularized cardiac extracellular matrix.

Yet another aspect of the disclosure encompasses embodiments of a pharmaceutically acceptable carrier generated from decellularized cardiac extracellular matrix.

Still another aspect of the disclosure encompasses embodiments of a therapeutic composition comprising a composition comprising a passenger chain oligonucleotide connected to a cell-penetrating peptide, wherein the cell-penetrating peptide and the passenger chain oligonucleotide can be connected by a linker covalently conjugated to the cell-penetrating peptide and to the passenger chain oligonucleotide, and wherein the composition can further comprise an miRNA oligonucleotide having a nucleotide sequence selectively hybridized to the passenger chain nucleotide sequence. and a pharmaceutically acceptable carrier.

In some embodiments of this aspect of the disclosure, the pharmaceutically acceptable carrier is a gel generated from decellularized cardiac extracellular matrix.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 shows the cell-penetrating peptide (CPP) Tat₄₇₋₅₇ modified miR-21(Tat-miR-21) construction according to the disclosure. Cysteine-conjugated Tat-peptide and amino-modified miR-21 passenger chain were synthesized. The Tat-miR-21 construct was produced through reaction with a succinimidyl 4-(N-maleimidomethyl)cyclohexane-1-carboxylate) (SMCC) linker. After annealing, the miR-21 mature oligonucleotide was base paired with the passenger chain.

FIG. 2 shows decellularization confirmed by hematoxylin-eosin (H&E) staining. After decellularization, the whole cell structure showed no nuclei (left panel) and the ECM to be porous (right panel).

FIG. 3 shows in vitro gelling of an extracellular (ECM) solution. By incubating the ECM solution in a 37° C. water bath for 30 min, injectable ECM hydrogel was formed with reduced fluidity and increased viscosity.

FIG. 4 shows SEM images of an ECM hydrogel of the disclosure. After gelling, microfibers were formed in ECM hydrogel.

FIG. 5 shows in vitro transfection of Tat-miR-21 in neonatal rat cardiomyocytes (NRCM) cells under normal culture. Tat-miR-21- or miR-21-containing medium was changed to culture NRCM cells for 16 h and the transfection efficacy was detected. Transfection agent Lip3000 was employed as the positive control.

FIG. 6 shows in vitro transfection of Tat-miR-21 in NRCM cells under reactive oxygen species (ROS) stress. ROS stress was introduced by incubating cells with 400 μM H₂O₂ before the transfection of Tat-miR-21.

FIG. 7 shows transfection of Tat-miR-21 rescue of NRCM cells having ROS injury. TUNEL staining was performed after ROS stress and the number of apoptotic cells was counted.

FIG. 8 shows that transfection of Tat-miR-21 promoted proliferation of NRCM cells after ROS injury. Cell proliferation was measured by immunostaining targeting Ki67. Ki67⁺ cell numbers were counted.

FIG. 9 shows epicardial spreading of ECM hydrogel after intrapericardial (iPC) injection in rat. H&E staining was performed 3 days after iPC injection. Injected ECM hydrogel was coating the epicardium tightly to form an overall cardiac patch.

FIG. 10 shows in vivo transfection of Tat-miR-21 into cardiomyocytes. Three days after iPC injection, transfection of Tat-miR-21 into cardiomyocytes was detected by targeting α-SA, the marker of cardiomyocytes.

FIG. 11 shows in vivo transfection of Tat-miR-21 into myofibroblast. Three days after iPC injection, transfection of Tat-miR-21 into myofibroblast was detected by targeting alpha smooth muscle actin (α-SMA), the marker of myofibroblast.

FIG. 12 shows in vivo transfection of Tat-miR-21 into endothelial cells. Three days after iPC injection, transfection of Tat-miR-21 into endothelial cells was detected by targeting vWF, the marker of endothelial cells.

FIG. 13 shows that iPC injection of Tat-miR-21 with ECM hydrogel promoted cardiac function recovery in rats. Myocardial infarcted (MI) rats received an iPC injection of Tat-miR-21 mixed in ECM hydrogel immediately after surgery, followed by echo measurement to show the baseline values. 4 weeks later, echo was measured again, and cardiac function indicators of left ventricular ejection fraction (LV-EF) and left ventricular fraction shortening (LV-FS) were calculated accordingly.

FIG. 14 illustrates that delivery of Tat-miR-21 improved cardiac morphology. Four weeks after intrapericardial injection of Tat-miR-21, the cardiac histology was analyzed by using Masson trichrome staining and Sirius-red staining. Improved cardiac morphology shown by reduced fibrotic scar was observed in Tat-miR-21-treated hearts. During the injection, extracellular matrix (ECM) hydrogel was used to prolong the retention of Tat-miR-21.

FIG. 15 illustrates that delivery of Tat-miR-21 reduced cardiac infarct size. Four weeks after intrapericardial injection of Tat-miR-21, the cardiac infarct size was calculated according to Masson trichrome staining. The infarct size was reduced after Tat-miR-21 treatment, and the scar thickness was increased.

### DETAILED DESCRIPTION

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

Before the embodiments of the present disclosure are described in detail, it is to be understood that, unless otherwise indicated, the present disclosure is not limited to particular materials, reagents, reaction materials, manufacturing processes, dimensions, frequency ranges, applications, or the like, as such can vary. It is also to be understood that the terminology used herein is for purposes of describing particular embodiments only, and is not intended to be limiting. It is also possible in the present disclosure that steps can be executed in different sequence, where this is logically possible. It is also possible that the embodiments of the present disclosure can be applied to additional embodiments involving measurements beyond the examples described herein, which are not intended to be limiting. It is furthermore possible that the embodiments of the present disclosure can be combined or integrated with other measurement techniques beyond the examples described herein, which are not intended to be limiting.

It should be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. Further, documents or references cited in this text, in a Reference List before the claims, or in the text itself; and each of these documents or references (“herein cited references”), as well as each document or reference cited in each of the herein-cited references (including any manufacturer's specifications, instructions, etc.) are hereby expressly incorporated herein by reference.

Prior to describing the various embodiments, the following definitions are provided and should be used unless otherwise indicated.

### Definitions

The term “cell” as used herein refers to an animal or human cell. The engineered cells of the disclosure can be removed (isolated) from a tissue and mixed with platelet-derived membrane vesicles without culturing of the cells, or after removal from a tissue or fluid of an animal or human may be cultured to increase the population size of the cells. If not immediately required for incorporation of the platelet-derived membrane vesicles, the animal or human cells may be maintained in a viable state either by culturing by serial passage in a culture medium or cryopreserved by methods well-known in the art. The cells may be obtained from the animal or human individual that has received an injury desired to be repaired by administration of the engineered cells of the disclosure or from a different individual.

Commercially available media may be used for the growth, culture and maintenance of mesenchymal stem cells. Such media include but are not limited to Dulbecco's modified Eagle's medium (DMEM). Components in such media that are useful for the growth, culture and maintenance of mesenchymal stem cells include but are not limited to amino acids, vitamins, a carbon source (natural and non-natural), salts, sugars, plant derived hydrolysates, sodium pyruvate, surfactants, ammonia, lipids, hormones or growth factors, buffers, non-natural amino acids, sugar precursors, indicators, nucleosides and/or nucleotides, butyrate or organics, DMSO, animal derived products, gene inducers, non-natural sugars, regulators of intracellular pH, betaine or osmoprotectant, trace elements, minerals, non-natural vitamins. Additional components that can be used to supplement a commercially available tissue culture medium include, for example, animal serum (e.g., fetal bovine serum (FBS), fetal calf serum (FCS), horse serum (HS)), antibiotics (e.g., including but not limited to, penicillin, streptomycin, neomycin sulfate, amphotericin B, blasticidin, chloramphenicol, amoxicillin, bacitracin, bleomycin, cephalosporin, chlortetracycline, zeocin, and puromycin), and glutamine (e.g., L-glutamine). Mesenchymal stem cell survival and growth also depends on the maintenance of an appropriate aerobic environment, pH, and temperature. Mesenchymal stem cells can be maintained using methods known in the art (see for example Pittenger et al., (1999) Science 284:143-147).

The term “cell penetrating peptide” as used herein refers to a peptide that facilitates the entry of said peptide, along with any molecule associated with the peptide, across one or more membranes to the interior of a cell.

CPPs are generally described as short peptides of between 10 and 30 amino acids either derived from proteins or from chimeric sequences. They are typically arginine-rich, amphipathic and lysine-rich, and hydrophobic and possess a net positive charge. CPPs are able to penetrate biological membranes, to trigger the movement of various biomolecules across cell membranes into the cytoplasm, and to improve their intracellular routing, thereby facilitating interactions with a target. CPPs are subdivided into two main classes, the first requiring chemical linkage with the cargo and the second involving the formation of stable, non-covalent complexes. CPPs have been attached to the N and C termini of payload proteins, and to intermediate positions using a variety of chemical conjugation strategies (e.g., targeting cysteine thiols). CPPs have been reported to favor the delivery of a large panel of cargos such as, but not limited to, plasmid DNA, oligonucleotide, siRNA, PNA, protein, peptide, liposome, nanoparticle, into a wide variety of cell types and in vivo models.

The initial interaction of CPP-cargo constructs with cellular membranes is through interactions with hydrophobic components and/or negatively charged groups (phospholipids, heparin sulfate proteoglycans) on the membrane surface. Uptake of CPP-bound payloads proceeds via binding to membrane and invagination. Depending on CPP tag, payloads can be targeted to internal compartments (nuclei, mitochondria) or cytoplasm. Once associated with the membrane surface, several translocation mechanisms can come into play, such as clathrin-dependent endocytosis, caveolin-dependent endocytosis, and macropinocytosis.

Since the initial discovery of the TAT peptide (TaTp), a variety of CPPs have been found to enable the transport of macromolecular cargoes to cells in culture and within living animals. A number of well characterized CPPs originated from the N or C termini of viral proteins; these include TATp, oligoarginines, MPG peptides, Pep1, and VP22. The TAT-CPP derived from the carboxy terminus of the dopamine transporter can enable the translocation of large cargoes.

The terms “microRNA” and “miRNA” as used herein refer to a small non-coding RNA molecule (containing about 22 nucleotides) found in plants, animals and some viruses, and which function in RNA silencing and post-transcriptional regulation of gene expression. miRNAs function via base-pairing with complementary sequences within mRNA molecules. As a result, these mRNA molecules are silenced, by one or more of cleavage of the mRNA strand into two pieces, destabilization of the mRNA through shortening of its poly(A) tail, and less efficient translation of the mRNA into proteins by ribosomes. miRNAs are derived from regions of RNA transcripts that fold back on themselves to form short hairpins.

The term “stem cell” as used herein refers to cells that have the capacity to self-renew and to generate differentiated progeny. The term “pluripotent stem cells” refers to stem cells that have complete differentiation versatility, i.e., the capacity to grow into any of the fetal or adult mammalian body's approximately 260 cell types. For example, pluripotent stem cells have the potential to differentiate into three germ layers: endoderm (e.g., blood vessels), mesoderm (e.g., muscle, bone and blood) and ectoderm (e.g., epidermal tissues and nervous system), and therefore, can give rise to any fetal or adult cell type. The term “cardiac stem cells” refers to stem cells obtained from or derived from cardiac tissue. The term “cardiosphere-derived cells (CDCs)” as used herein refers to undifferentiated cells that grow as self-adherent clusters from subcultures of postnatal cardiac surgical biopsy specimens. CDCs can express stem cell as well as endothelial progenitor cell markers, and typically possess properties of adult cardiac stem cells. For example, human CDCs can be distinguished from human cardiac stem cells in that human CDCs typically do not express multidrug resistance protein 1 (MDR1; also known as ABCB1), CD45 and CD133 (also known as PROM1). CDCs are capable of long-term self-renewal, and can differentiate in vitro to yield cardiomyocytes or vascular cells after ectopic (dorsal subcutaneous connective tissue) or orthotopic (myocardial infarction) transplantation in SCID beige mouse.

The terms “cardiac progenitor cells” and “cardiac stem cells” as used herein can refer to a population of progenitor cells derived from human heart tissue. In some aspects, the present disclosure, of the cardiac progenitor (stem) cells, at least 3%, 5%, 7%, 10%, 12%, or 15%, e.g., 3-50%, 3-20%, 3-10%, 5-30%, 5-10%, etc., of the cells express Isl1. In some aspects, CPCs comprise about 10%, 15%, 20%, 30%, 40%, or 50%, e.g., 10-50%, 10-40%, 10-30%, 15-40%, etc., GATA4 expressing cells. In some aspects, cardiac stem cells comprise about 5%, 8%, 10%, 13%, or 15%, e.g., 8-15%, 5-15%, 5-13%, etc., NKX2.5 expressing cells. Before fusion with platelet membrane vesicles by the methods of the present disclosure, cardiac stem cells are unmodified cells in that recombinant nucleic acids or proteins have not been introduced into them or the Sca-1⁺, CD45-cell from which it is derived. As such, cardiac stem cells as isolated from cardiac tissue are non-transgenic, or in other words have not been genetically modified. For example, expression of genes such as Isl1, GATA4, and NKX2.5 in CPCs is from the endogenous gene. Cardiac stem cells may comprise Sca-1+, CD45-cells, c-kit⁺ cells, CD90⁺ cells, CD133⁺ cells, CD31⁺ cells, Flk1⁺ cells, GATA4⁺ cells, or NKX2.5+ cells, or combinations thereof. In some aspects, cardiac stem cells comprise about 50% GATA4 expressing cells. In some aspects, cardiac stem cells comprise about 15% NKX2.5 expressing cells. Cardiac stem cells can replicate and are capable of differentiating into endothelial cells, cardiomyocytes, smooth muscle cells, and the like.

The term “cardiac cells” as used herein refers to any cells present in the heart that provide a cardiac function, such as heart contraction or blood supply, or otherwise serve to maintain the structure of the heart. Cardiac cells as used herein encompass cells that exist in the epicardium, myocardium or endocardium of the heart. Cardiac cells also include, for example, cardiac muscle cells or cardiomyocytes; cells of the cardiac vasculatures, such as cells of a coronary artery or vein. Other non-limiting examples of cardiac cells include epithelial cells, endothelial cells, fibroblasts, cardiac conducting cells and cardiac pacemaking cells that constitute the cardiac muscle, blood vessels and cardiac cell supporting structure.

The term “cardiac function” as used herein refers to the function of the heart, including global and regional functions of the heart. The term “global” cardiac function as used herein refers to function of the heart as a whole. Such function can be measured by, for example, stroke volume, ejection fraction, cardiac output, cardiac contractility, etc. The term “regional cardiac function” refers to the function of a portion or region of the heart. Such regional function can be measured, for example, by wall thickening, wall motion, myocardial mass, segmental shortening, ventricular remodeling, new muscle formation, the percentage of cardiac cell proliferation and programmed cell death, angiogenesis and the size of fibrous and infarct tissue. Techniques for assessing global and regional cardiac function are known in the art. For example, techniques that can be used to measure regional and global cardiac function include, but are not limited to, echocardiography (e.g., transthoracic echocardiogram, transesophageal echocardiogram or 3D echocardiography), cardiac angiography and hemodynamics, radionuclide imaging, magnetic resonance imaging (MRI), sonomicrometry and histological techniques.

The term “cardiac tissue” as used herein refers to tissue of the heart, for example, the epicardium, myocardium or endocardium, or portion thereof, of the heart. The term “injured” cardiac tissue as used herein refers to a cardiac tissue that is, for example, ischemic, infarcted, reperfused, or otherwise focally or diffusely injured or diseased. Injuries associated with a cardiac tissue include any areas of abnormal tissue in the heart, including any areas caused by a disease, disorder or injury and includes damage to the epicardium, endocardium and/or myocardium. Non-limiting examples of causes of cardiac tissue injuries include acute or chronic stress (e.g., systemic hypertension, pulmonary hypertension or valve dysfunction), atheromatous disorders of blood vessels (e.g., coronary artery disease), ischemia, infarction, inflammatory disease and cardiomyopathies or myocarditis.

The terms “generate”, “generation”, and “generating” as used herein shall be given their ordinary meaning and shall refer to the production of new cells in a subject and optionally the further differentiation into mature, functioning cells. Generation of cells may comprise regeneration of the cells. Generation of cells comprises improving survival, engraftment and/or proliferation of the cells.

The terms “regenerate,” “regeneration” and “regenerating” as used herein refer to the process of growing and/or developing new cardiac tissue in a heart or cardiac tissue that has been injured, for example, injured due to ischemia, infarction, reperfusion, or other disease. Tissue regeneration may comprise activation and/or enhancement of cell proliferation. Cardiac tissue regeneration comprises activation and/or enhancement of cell migration.

The term “cell therapy” as used herein refers to the introduction of new cells into a tissue in order to treat a disease and represents a method for repairing or replacing diseased tissue with healthy tissue.

The term “derived from” as used herein refers to cells or a biological sample (e.g., blood, tissue, bodily fluids, etc.) and indicates that the cells or the biological sample were obtained from the stated source at some point in time. For example, a cell derived from an individual can represent a primary cell obtained directly from the individual (i.e., unmodified). In some instances, a cell derived from a given source undergoes one or more rounds of cell division and/or cell differentiation such that the original cell no longer exists, but the continuing cell (e.g., daughter cells from all generations) will be understood to be derived from the same source. The term includes directly obtained from, isolated and cultured, or obtained, frozen, and thawed. The term “derived from” may also refer to a component or fragment of a cell obtained from a tissue or cell.

The term “isolating” or “isolated” when referring to a cell or a molecule (e.g., nucleic acids or protein) indicates that the cell or molecule is or has been separated from its natural, original or previous environment. For example, an isolated cell can be removed from a tissue derived from its host individual, but can exist in the presence of other cells (e.g., in culture), or be reintroduced into its host individual.

The term “culturing” as used herein refers to growing cells or tissue under controlled conditions suitable for survival, generally outside the body (e.g., ex vivo or in vitro). The term includes “expanding,” “passaging,” “maintaining,” etc. when referring to cell culture of the process of culturing. Culturing cells can result in cell growth, differentiation, and/or division.

The term “disaggregating” includes separating, dislodging, or dissociating cells or tissue using mechanical or enzymatic disruption to isolate single cells or small clusters of cells. In some instances, enzymatic disruption can be replaced with one of more enzyme alternatives having substantially the same effect as the enzyme.

The term “tissue injury” as used herein refers to damage to a vascularized tissue of an animal or human, wherein the damage is adjacent to, or in close proximity to, a blood vessel that has also undergone injury, and in particular loss of endothelial cells lining the lumen of the blood vessel. For example, but not intended to be limiting, vascular ischemia can result in both loss of vascular endothelial cells to expose the underlying subendothelial matrix. The loss of adequate blood flow can result in loss of cell viability in such as cardiac tissue, brain or neurological tissue that is in contact with the occluded blood vessel.

The term “endothelial cell” refers to a cell necessary for the formation and development of new blood vessel from pre-existing vessels (e.g., angiogenesis). Typically, endothelial cells are the thin layer of cells that line the interior surface of blood vessels and lymphatic vessels. Endothelial cells are involved in various aspects of vascular biology, including atherosclerosis, blood clotting, inflammation, angiogenesis, and control of blood pressure.

The term “smooth muscle cell” refers to a cell comprising non-striated muscle (e.g., smooth muscle). Smooth muscle is present within the walls of blood vessels, lymphatic vessels, cardiac muscle, urinary bladder, uterus, reproductive tracts, gastrointestinal tract, respiratory tract, and iris of the eye.

The term “cardiomyocyte cell” refers to a cell comprising striated muscle of the walls of the heart. Cardiomyocytes can contain one or more nuclei.

The term “cardiosphere” refers to a cluster of cells derived from heart tissue or heart cells. In some instances, a cardiosphere includes cells that express stem cell markers (e.g., c-Kit, Sca-1, and the like) and differentiating cells expressing myocyte proteins and the gap protein (connexin 43).

The term “autologous” refers to deriving from or originating in the same subject or patient. An “autologous transplant” refers to collection (e.g., isolation) and re-transplantation of a subject's own cells or organs. In some instances, an “autologous transplant” includes cells grown or cultured from a subject's own cells. For example, in the methods of the present disclosure, the cardiac stem cells may be derived from a cardiac tissue sample excised from the heart of the patient to be treated, cultured, engineered to be fused with platelet membrane vesicles according to the methods of the disclosure and then administered to the same patient for the treatment of a cardiovascular injury therein.

The term “allogeneic” refers to deriving from or originating in another subject or patient. An “allogeneic transplant” refers to collection (e.g., isolation) and transplantation of the cells or organs from one subject into the body of another. In some instances, an “allogeneic transplant” includes cells grown or cultured from another subject's cells.

The term “transplant” as used herein refers to cells, e.g., cardiac progenitor cells, introduced into a subject. The source of the transplanted material can include cultured cells, cells from another individual, or cells from the same individual (e.g., after the cells are cultured, enriched, or expanded ex vivo or in vitro).

The terms “treatment,” “therapy,” “amelioration” and the like refer to any reduction in the severity of symptoms. As used herein, the terms “treat” and “prevent” are not intended to be absolute terms. Treatment can refer to any delay in onset, amelioration of symptoms, improvement in patient survival, repair/regeneration of heart tissue or blood vessels, increase in survival time or rate, etc. The effect of treatment can be compared to an individual or pool of individuals not receiving the treatment. In some instances, the effect can be the same patient prior to treatment or at a different time during the course of therapy. In some aspects, the severity of disease, disorder or injury is reduced by at least 10%, as compared, e.g., to the individual before administration or to a control individual (e.g., healthy individual or an individual no longer having the disease, disorder or injury) not undergoing treatment. In some instances, the severity of disease, disorder or injury is reduced by at least 20%, 25%, 50%, 75%, 80%, or 90%. In some cases, the symptoms or severity of disease are no longer detectable using standard diagnostic techniques.

The terms “subject,” “patient,” “individual” and the like are used interchangeably and refer to, except where indicated, mammals such as humans and non-human primates, as well as rabbits, rats, mice, dogs, cats, goats, pigs, cows, and other mammalian species. The term does not necessarily indicate that the subject has been diagnosed with a particular disease, but typically refers to an individual under medical supervision.

The term “contacting” as used herein refers to the placement in direct physical association, including both a solid and liquid form. Contacting can occur in vitro with isolated cells or in vivo by administering to a subject. In an example, a cell, such as a cardiac cell, may be contacted with or exposed to a therapeutically effective concentration of an agent, including one of the disclosed compositions.

The term “polypeptide” as used herein refers to a polymer in which the monomers are amino acid residues which are joined together through amide bonds. When the amino acids are alpha-amino acids, either the L-optical isomer or the D-optical isomer can be used. The terms “polypeptide” or “protein” as used herein are intended to encompass any amino acid sequence and include modified sequences such as glycoproteins. The term “polypeptide” is intended to cover naturally occurring proteins, as well as those which are recombinantly or synthetically produced.

The term “Protein Transduction Domain (PTD)” as used herein refers to a family of polypeptides that facilitate protein transduction across membranes in a receptor-independent manner. This phenomena was originally described for the human immunodeficiency virus (HIV)-encoded transactivator of transcription (TAT) protein, which was shown to cross membranes and initiate transcription. The portion of the TAT protein required for the transduction of the protein was only an 11 amino acid polypeptide: tyrosine (Y), glycine (G), arginine (R), lysine (K), lysine (K), arginine (R), arginine (R), glutamine (Q), arginine (R), arginine (R), arginine (R) (YGRKKRRQRRR; SEQ ID NO: 1). When fused with other proteins, the TAT peptide has been demonstrated to deliver these proteins, varying in size from 15 to 120 kDa, into cells in tissue culture.

Other TAT polypeptide sequences that may be used in the compositions of the disclosure include, but are not limited to RKKRRQRRR (SEQ ID NO: 4); KKKKKKKKK (SEQ ID NO: 5); or RRRRRRRRR (SEQ ID NO: 6). The PDG polypeptides provided herein need not comprise a TAT peptide sequence having 100% identity to SEQ ID NO: 1, 4, 5, or 6. The current disclosure contemplates use of a modified or variant TAT peptide in which one or more amino acids differ from one of the TAT peptide sequences provided herein. However, the modified or variant TAT peptide contemplated for use retains the capacity to facilitate protein transduction across membranes. Methods of preparing TAT fusion proteins and expression vectors comprising TAT fusion proteins are well known in the art.

Other PTDs are known in the art and can be used in the compositions and methods described herein. A number of artificial peptides also are known to function as PTDs, such as poly-arginine, poly-lysine and others.

### Abbreviations

NRCM, neonatal rat cardiomyocyte; PBS, phosphate-buffered saline; LVEF, left ventricular ejection fraction; MI, myocardial infarction; vWF, von Willebrand factor; CM, cardiomyocyte; CF, cardiac fibroblast (CF); EC, endothelial cell; DMEM, Dulbecco's modified Eagle's medium

### Discussion

Successful entrance of microRNA-21-5p (miR-21) into cells is the basis for its therapeutic efficacy, especially in repair of damaged cardiac tissue. The inherent natural ability of cell membranes to exclude exogenous nucleic acids makes the transfection of microRNA a key issue in developing microRNA therapy.

Derived from the transmembrane domain of membrane-integrating protein, a cell penetrating peptide is able to readily cross the cell membrane. Provided, therefore, is the Tat-miR-21 conjugate of the disclosure, in which the Tat is a cell penetrating peptide. Highly efficient transfection of Tat-miR-21 into cells was found for both in vitro and in vivo situations. In a rat model of myocardial infarction, treatment with the Tat-miR-21 conjugate significantly improved the functional outcomes. Accordingly, Tat modification of miRNA is significantly advantageous for the efficient delivery of this or any other desired miRNA to a cell. The delivery of such miRNA compositions to a cell and the transfection thereof can further be assisted by admixing with a hydrogel that can be formed from a decellularized preparation of an extracellular matrix such as, but not limited to, a cardiac tissue extracellular matrix.

The present disclosure, therefore, encompasses compositions and methods useful in regenerating damaged cardiac tissue by delivering to the site of injury a therapeutic composition comprising such as, but not limited to, an miRNA that reduces the expression of phosphatase and tensin homolog (PTEN) that is over-expressed in normal cardiac stromal cells compared to the expression level in stromal cells from injured cardiac tissue. However, the compositions and methods of the disclosure may be generally applied to deliver an miRNA to a cell or tissue such as, but not limited to, a neuron, a smooth muscle cell, or a tumor cell. Provided, therefore, are compositions that comprise an miRNA species such as, but not limited to, hsa-miR-21-5p (SEQ ID NO: 2) conjugated to a carrier peptide that facilitates the entry of the miRNA into cells such as cardiac cells.

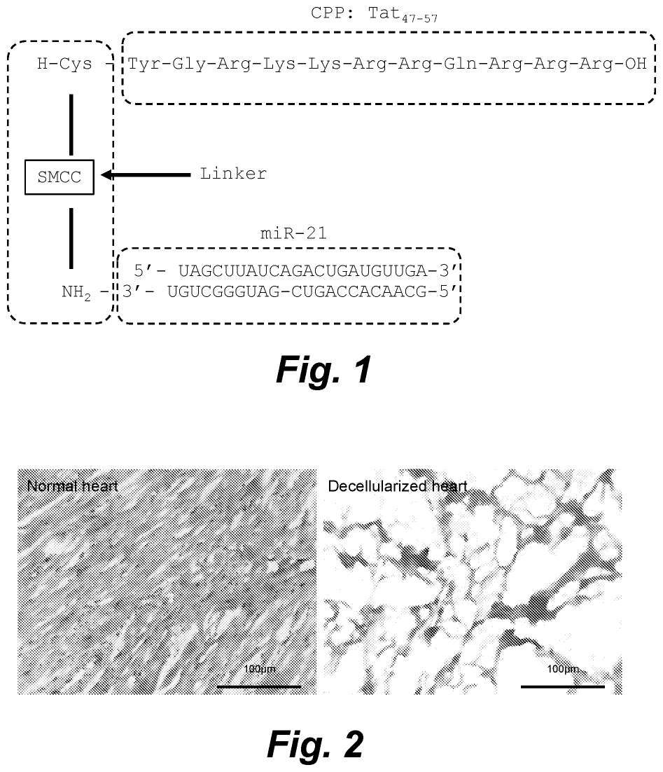

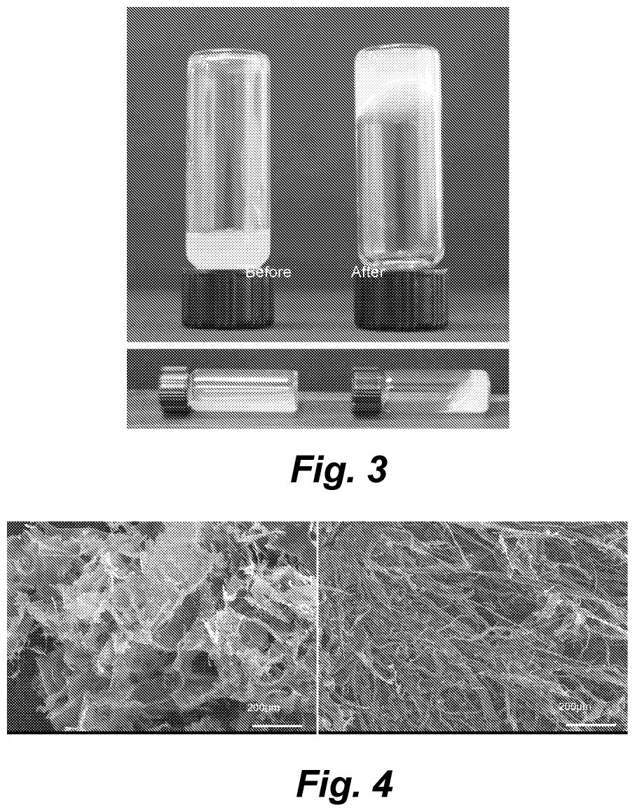

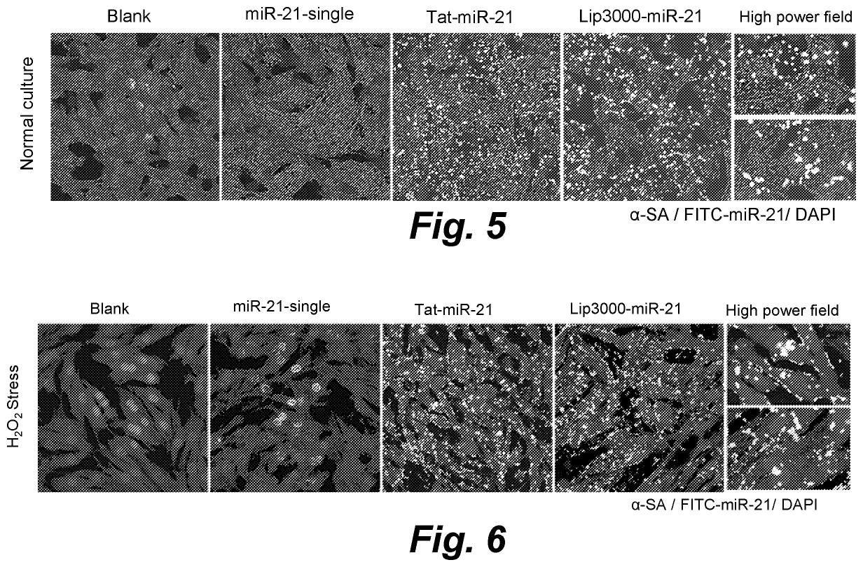

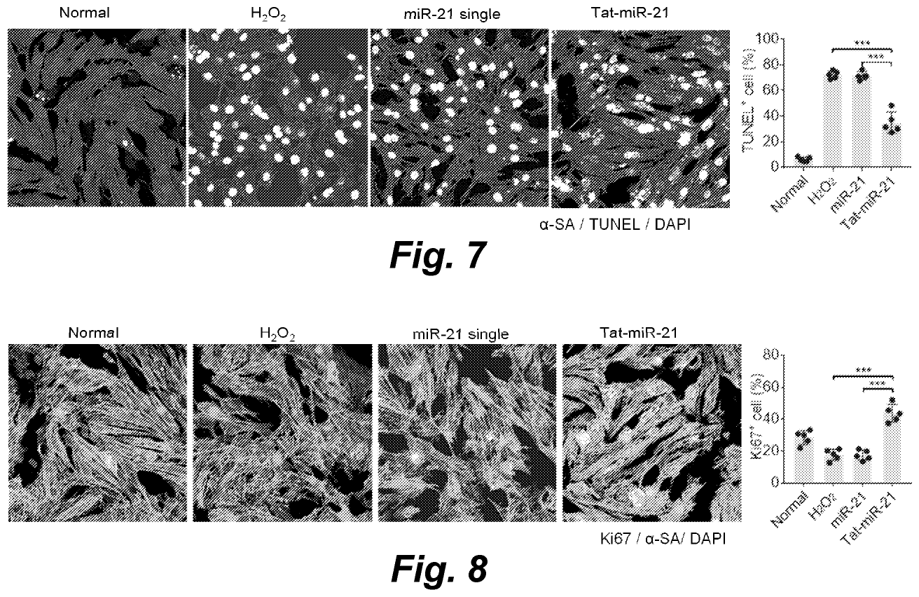

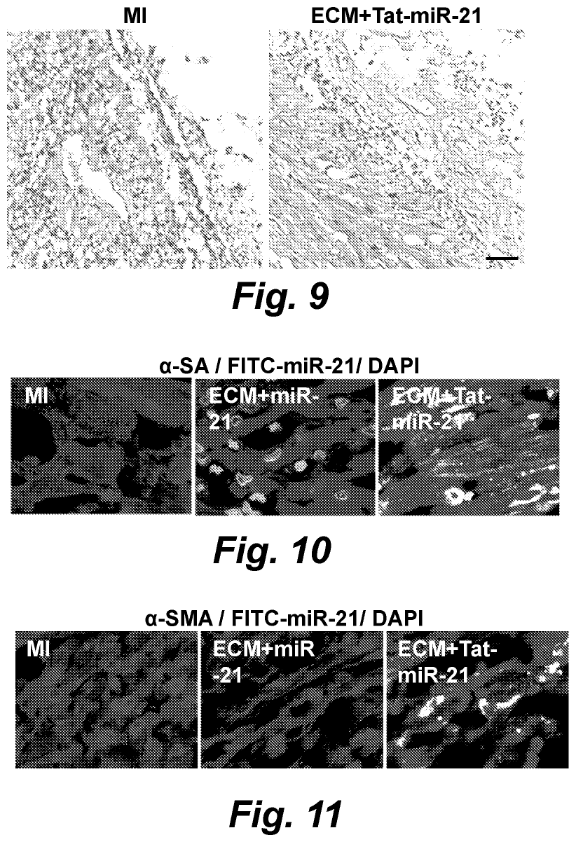

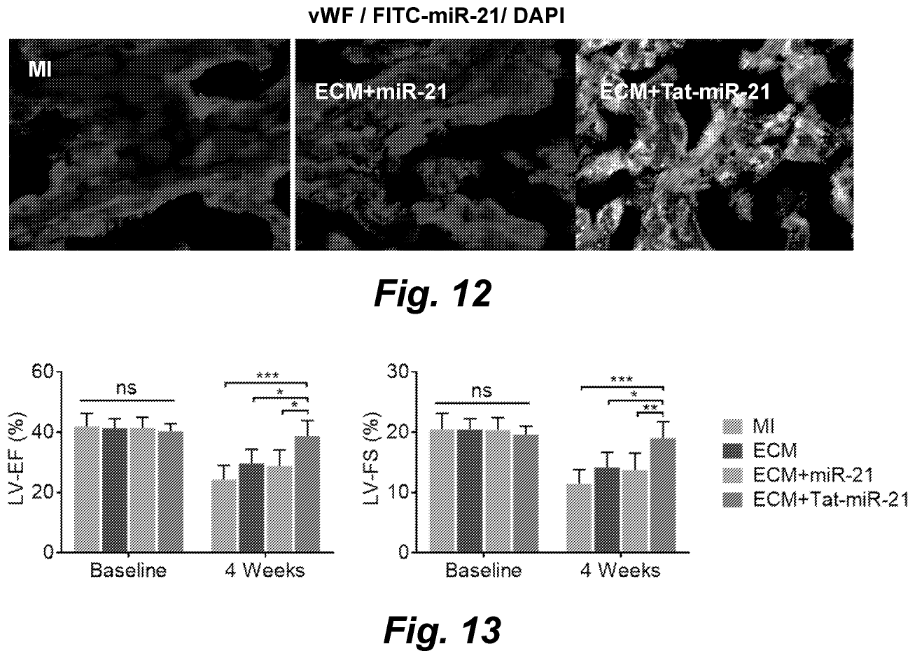

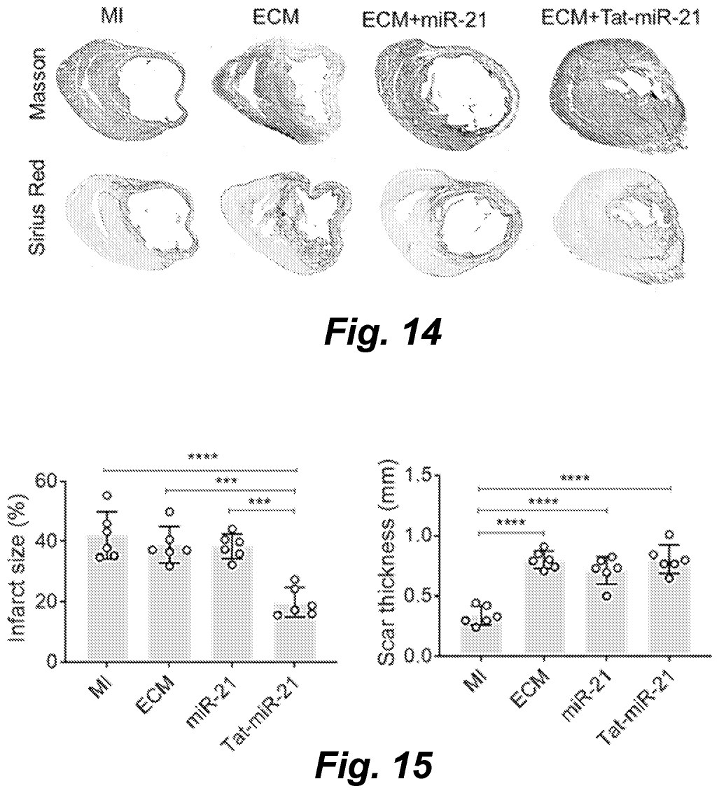

In the embodiments of the disclosure the membrane-transporting peptide may be any that is known in the art, However, a most advantageous such carrier peptide-based composition uses the cell penetrating peptide (CPP) HIV-1 transactivator (Tat) protein (47-57) having the amino acid sequence SEQ ID NO: 1 and which is conjugated to a passenger chain oligonucleotide substantially complementary to the nucleotide sequence of an miRNA. For use as a means of delivery to a cell, and in particular to isolated or cultured cardiac cells or cardiac cells in the heart tissue of an animal or human subject, the miRNA oligonucleotide desired to be delivered to a target cell or tissue is annealed to the passenger chain oligonucleotide, as shown for example in FIG. 1. It is, however considered possible to select an oligonucleotide for conjugation to a carrier peptide, the oligonucleotide having a sequence capable of hybridizing to a selected mRNA desired to be delivered to a target tissue of cell type.

While the target cells intended to receive the miRNA may be transfected with the miRNA-cell penetrating peptide composition of the disclosure using methods well-known in the art, it has been found surprisingly advantageous to admix the miRNA-passenger chain-cell-penetrating peptide compositions of the disclosure with a hydrogel that has been generated by suspending a decellularized extracellular matrix in a physiologically acceptable aqueous composition such as, but not limited to, deionized water. The decellularize extracellular matrix can be isolated from the targeted tissue that may allow an increase in the targeting of the composition to the tissue or it may be a hydrogel produced with a decellularized extracellular matrix not from the target tissue but still allowing effective delivery of the peptide-oligonucleotide composition to the tissue and cells.

The compositions of the disclosure, therefore, are conjugates of a passenger oligonucleotide (RNA or DNA) and a cell membrane penetrating peptide (CMPP). The two passenger oligonucleotide and the CMPP may be linked by a linker such as (for example) SMCC.

One aspect of the disclosure, therefore, encompasses embodiments of a composition that can comprise a passenger chain oligonucleotide connected to a cell-penetrating peptide.

In some embodiments of this aspect of the disclosure, the cell-penetrating peptide and the passenger chain oligonucleotide can be connected by a linker.

In some embodiments of this aspect of the disclosure, the linker can be covalently conjugated to the cell-penetrating peptide and to the passenger chain oligonucleotide

In some embodiments of this aspect of the disclosure, the composition can further comprise an miRNA oligonucleotide having a nucleotide sequence selectively hybridizable to the passenger chain nucleotide sequence.

In some embodiments of this aspect of the disclosure, the passenger chain oligonucleotide can have an amino group at the 3′ terminus thereof.

In some embodiments of this aspect of the disclosure, the cell-penetrating peptide can be HIV-1 transactivator (Tat) Protein (47-57) having the amino acid sequence SEQ ID NO: 1.

In some embodiments of this aspect of the disclosure, the HIV-1 transactivator (Tat) Protein (47-57) can further comprise a cysteine residue conjugated to the amino terminus of the HIV-1 transactivator (Tat) Protein (47-57).

In some embodiments of this aspect of the disclosure, the linker can be (succinimidyl 4-(N-maleimidomethyl)cyclohexane-1-carboxylate) (SMCC).

In some embodiments of this aspect of the disclosure, the passenger chain oligonucleotide can have the nucleotide sequence of SEQ ID NO: 2.

In some embodiments of this aspect of the disclosure, the HIV-1 transactivator (Tat) Protein (47-57) can further comprise a cysteine residue conjugated to the amino terminus of the peptide SEQ ID NO: 1, and wherein the cysteine residue can be further conjugated to a linker, wherein the linker can be (succinimidyl 4-(N-maleimidomethyl)cyclohexane-1-carboxylate) (SMCC), and wherein the linker can be further conjugated to the passenger chain oligonucleotide, the passenger chain oligonucleotide having an amino group at the 3′ terminus thereof.

In some embodiments of this aspect of the disclosure, the composition has the formula I:

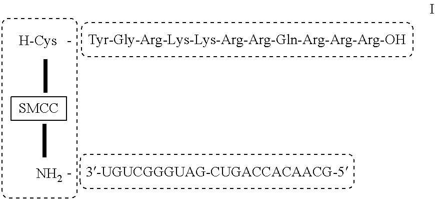

In some embodiments of this aspect of the disclosure, the miRNA can be miRNA-21 and can have a nucleotide sequence of SEQ ID NO: 3.

In some embodiments of this aspect of the disclosure, the composition can have the formula II:

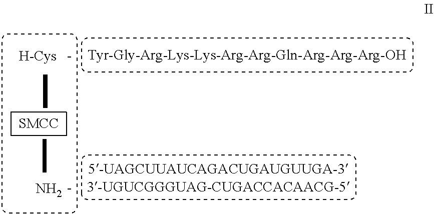

Another aspect of the disclosure encompasses embodiments of a method of delivering an miRNA to a cell, the method comprising contacting a cell with a composition comprising a passenger chain oligonucleotide connected to a cell-penetrating peptide, wherein the cell-penetrating peptide and the passenger chain oligonucleotide can be connected by a linker covalently conjugated to the cell-penetrating peptide and to the passenger chain oligonucleotide, and wherein the composition can further comprise an miRNA oligonucleotide having a nucleotide sequence selectively hybridized to the passenger chain nucleotide sequence.

In some embodiments of this aspect of the disclosure, the cell can be a cardiac cell.

In some embodiments of this aspect of the disclosure, the cardiac cell can be a cardiomyocyte.

In some embodiments of this aspect of the disclosure, the composition can be administered to a human or animal subject, and wherein the composition is admixed with a pharmaceutically acceptable carrier.

In some embodiments of this aspect of the disclosure, the pharmaceutically acceptable carrier can be a hydrogel generated from decellularized cardiac extracellular matrix.

Yet another aspect of the disclosure encompasses embodiments of a pharmaceutically acceptable carrier generated from decellularized cardiac extracellular matrix.

Still another aspect of the disclosure encompasses embodiments of a therapeutic composition comprising a composition comprising a passenger chain oligonucleotide connected to a cell-penetrating peptide, wherein the cell-penetrating peptide and the passenger chain oligonucleotide can be connected by a linker covalently conjugated to the cell-penetrating peptide and to the passenger chain oligonucleotide, and wherein the composition can further comprise an miRNA oligonucleotide having a nucleotide sequence selectively hybridized to the passenger chain nucleotide sequence. and a pharmaceutically acceptable carrier.

In some embodiments of this aspect of the disclosure, the pharmaceutically acceptable carrier is a gel generated from decellularized cardiac extracellular matrix.

As mentioned above, compounds of the present disclosure and pharmaceutical compositions can be used in combination of one or more other therapeutic agents for treating viral infection and other diseases. For example, compounds of the present disclosure and pharmaceutical compositions provided herein can be employed in combination with other anti-viral agents to treat viral infection.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### EXAMPLES

### Example 1

Cell Culture and Exosome Isolation: Diseased heart tissues were received from patients who underwent heart transplantation after heart failure. Healthy heart tissues were received from normal, non-damaged, human donor hearts. Cardiac cells were separated and cultured as previously described (Makkar et al., (2012) Lancet 379: 895-904). In brief, myocardial tissues harvested from donors' hearts were separated and washed with PBS, then cut into smaller 2 mm³ fragments, and partially enzymatically digested at 37° C. with collagenase type IV (C1889, Sigma-Aldrich). The tissue samples were plated onto fibronectin-coated petri dishes in Iscove's modified Dulbecco's medium (IMDM, Gibco) infused with 20% fetal bovine serum (FBS; Corning). After 3-5 days, cardiac cells began to grow from the explants.

Exosomes were isolated from the conditioned medium of the cardiac cells. Passage 1-3 cardiac cells were cultured to 80% confluency. The medium was then switched to serum-free IMDM and conditioned for 14 days. Exosomes were then isolated from the conditioned medium by ultrafiltration (Vandergriff et al., (2018) Theranostics 8: 1869-1878; Vandergriff et al., (2015) Stem Cells Int. 2015: 960926). In brief, conditioned medium was filtered through 0.22 μm Steriflip filters to remove cellular debris and large vesicles. The filtrate was then added to Amicon Ultra-15 100 kDa filters (SCGP00525, Millipore) to centrifuge at 5,000×g for 5 min. The flow-through was discarded. The concentrated exosomes were collected and washed with PBS three times, and stored at −80° C. Labeling was performed using 10 μM Dil (V22889, Thermo Fisher Scientific)

### Example 2

Flow Cytometry: Flow cytometry was performed to examine the antigenic phenotypes of cardiac cells. Cells were incubated with antibodies against CD90 (555595, BD), CD105 (ab11414, Abcam), CD31 (555445, BD), CD 34 (ab81289, Abcam), CD 45 (555482, BD), and c-kit (550412, BD) for 60 mins at 4° C. Both unstained and isotype controls (555748, 559320, BD) were used as negative controls. Flow cytometry was conducted with a CytoFlex Flow Cytometer (Beckman Coulter) and data were analyzed with FCS Express software (De Novo).

### Example 3

Construction of Tat-miR-21: Sulfo-SMCC (sulfosuccinimidyl 4-(N-maleimidomethyl)cyclohexane-1-carboxylate) (Sulfo-SMCC) was used as the linker of the Tat-peptide and the miR-21. Firstly, sulfo-SMCC, cysteine-modified Tat-peptide, amino-modified mi-21 passenger chain as well as mi-21 mature chain were individually synthesized. Then the SMCC-conjugated Tat-peptide was produced by adding 100 μl of 10 mg/ml sulfo-SMCC solution (50 mM) into 500 μl 10 mg/ml Tat-peptide solution (6 mM) to make a mixture of sulfo-SMCC with Tat-peptide in a ratio of 1.7:1, which was placed at 4° C. overnight with shaking at 500 rpm. Annealed miR-21 solution was added into the SMCC-Tat solution with a concentration ratio of 1:1, and the mixture was placed at room temperature for 2 hours.

### Example 4

Preparation of Extracellular Matrix (ECM) hydrogel: Pig heart tissues were cut into pieces of 2 mm in thickness, and rinsed with deionized water. Decellularization was by immersing the tissues in 1% SDS in PBS for 4-5 days, until the tissue was white, then the tissue was placed in 1% Triton X-100 and stir for 30 min for final cell removal. The decellularized heart tissue was washed with deionized (DI) water for more than 24 h to remove detergent.

To produce the ECM hydrogel, the decellularized ECM was lyophilized and milled into a fine powder, followed by enzymatic digestion using pepsin dissolved in 0.1M HCl for at least 48 h. The pepsin-matrix ratio was 1:10 and stirring continued during digestion. Finally, the pH was adjusted to 7.4 with NaOH on ice and the ECM solution was adjusted to 6 mg/mL.

Gelling could be induced by incubating the ECM solution at 37° C. for about 30 min, forming the ECM hydrogel. Confirm of decellularization was by cryo-section. After deionized water washing, the tissues were immersed in 30% sucrose, and further embedded with Optimal Cutting Temperature compound (OCT) (10.24% polyvinyl alcohol; 4.26% polyethylene glycol; 85.5% non-reactive ingredients). 5 μm cryosections were cut for H&E staining.

### Example 5

Neonatal Rat Cardiomyocytes (NRCM) isolation and culture: The heart tissues of rat pups were collected and each chopped into 4-6 pieces. After washing with fresh HBSS, the tissues were transferred into 0.1% trypsin solution and digested overnight at 4° C. The supernatant was collected and the heart tissues were further digested repeatedly with collagenase until dissolved. All the cell-containing supernatant was centrifuged at 410 rcf for 10 min to collect cells. After washing with cold HBSS and filtering through a 40 μm strainer, the cell pellet was harvested and further cultured with 10% FBS-containing Iscove's Modified Dulbecco's Medium (IMDM).

### Example 6

H₂O₂ stress and in vitro transfection: NRCM cells were cultured under normal conditions for up to five days until the cells were 80% confluence. H₂O₂-containing (400 μM) medium was then prepared and added to mimic the reactive oxygen species (ROS) stress of myocardial infarction (MI) injury.

This stress was allowed for 4 h, after which fresh medium with either Tat-miR-21 conjugate or miR-21 alone (both at a concentration of 800 nM) was added and further incubated for 16 h. The transfection agent Lip3000 was employed as a positive control and the load dose of miR-21 was 800 nM.

### Example 7

Rat model of myocardial infarction and intrapericardial injection: Briefly, the animal was anesthetized through IP injection of Ketamine-Xylazine (KX) in a dose of 100 mg/kg and 5 mg/kg respectively, followed by ventilation, and thoracotomy. Then the heart left anterior descending artery (LAD) was ligated with a 6-0 suture with the pericardium entirely preserved. Infarction was confirmed by a pale change in the apex. During the injection, the suture knot was gently held with forceps and hydrogel, with or without therapeutics, was injected into the pericardial cavity. The injection volume was 100 μL. After injection, the chest was closed and the model was allowed to recovery.

### Example 8

Cardiac function measurement: Cardiac function was measured at indicated time points. After anesthesia with inhalation of isoflurane, the animals were fixed to the operating plate with the body temperature maintained at 37° C. Then the M-mode cardiac movement was observed and recorded with an echo machine equipped with a 40 MHz transducer. The left ventricular dimension at both diastole (LVIDd) and systole (LVIDs) were measured, and accordingly, the values of ejection fraction (EF), fraction shortening (FS), and LV volume at end diastole (EDV) and systole (ESV) were calculated. Five continuous cardiac cycles were measured for each animal.

### Example 9

Immunocytochemistry: Cells were fixed with 4% PFA for 15 min at room temperature (RT), followed by washing twice with PBS. Blocking serum was then added and incubated at RT for 1 hour to block the non-specific staining. After that the primary antibody (Ki67, α-SA) working solution was added and incubated overnight at 4° C. After washing with PBS, the corresponding secondary antibody was incubated. DAPI was used to stain the nucleus. TUNEL staining was by using a labelling kit (Promega, G3250), and after reaction, α-SA staining was performed.

### Example 10

Histological analysis: At indicated time points, the animals were scarified with inhalation of CO₂, followed by intraventricular perfusion of chilled saline and 4% PFA. An excised heart wrapped with pericardium was harvested and immersed in 4% PFA overnight. After washing with PBS for twice, the heart was placed into 30% glucose, followed by embedding with OCT and cryosectioned into a series of sections of 5 μm thickness and stored at −20° C.

H&E staining, Masson trichrome staining was performed by standard protocols. Sections were fixed in Bouin's solution overnight, followed by water washing and staining with hematoxylin. After differentiation, the sections were placed in eosin solution for staining.

For immunostaining cryosections were immersed in absolute methanol for 10 min at RT, followed by air drying and water washing to remove OCT. Sections were blocked with DAKO blocking solution mixed with 0.1% saponin for 1 hour at RT. Primary antibody diluted with blocking solution was then dropped to cover the tissues and incubated at 4° C. overnight, following by three washes with PBS. Secondary antibody was then added and incubated for 2 h at RT. After washing with PBS, the sliders were mounted with DAPI Fluoromount-G (Southern Biotech, 0100-20).

### Example 11

Antibodies and kits: Antibodies against Ki67 (ab16667), α-Sarcomeric Actinin (SA, ab9465), vWF (ab6994), α-SMA (ab32575) as well as Alexa Fluo 594 or 488 conjugated Goat anti Rabbit or mouse secondary antibodies were from Abcam. TUNEL kit was from Promega (G3250). Protein labelling probe and DiD were from Thermo Fisher.

### Example 12

Images acquisition and statistical analyses: For cell experiments, triplicated wells were introduced. For in vivo study, the number of animals in each group were randomly determined. For quantification, more than five images were acquired in each animal/group, and accordingly the counts were acquired.

Statistical analyses were performed by using Graph Pad Prism 7 and data was expressed as mean±SD. Comparisons between two groups were performed with Student t-test, while for multiple groups comparison, one-way ANOVA and two-way ANOVA were introduced. p<0.05 was recognized as significant difference.

TABLE 2
Example 13

| | Accession | |
|---|---|---|
| miRNA | Number^{a} | Sequence |
| hsa-miR- | MIMAT0000076 | UAGCUUAUCAGACUGAUGUUGA |
| 21-5p | | (SEQ ID NO: 3) |

^{a}miRBase accession number

What is claimed:

1. A composition comprising a passenger chain oligonucleotide connected to a cell-penetrating peptide.

2. The composition of claim 1, wherein the cell-penetrating peptide and the passenger chain oligonucleotide are connected by a linker.

3. The composition of claim 2, wherein the linker is covalently conjugated to the cell-penetrating peptide and to the passenger chain oligonucleotide

4. The composition of claim 1, wherein the composition further comprises an miRNA oligonucleotide having a nucleotide sequence selectively hybridizable to the passenger chain nucleotide sequence.

5. The composition of claim 1, wherein the passenger chain oligonucleotide has an amino group at the 3′ terminus thereof.

6. The composition of claim 1, wherein the cell-penetrating peptide is HIV-1 transactivator (Tat) Protein (47-57) having the amino acid sequence SEQ ID NO: 1.

7. The composition of claim 6, wherein the HIV-1 transactivator (Tat) Protein (47-57) further comprises a cysteine residue conjugated to the amino terminus of the HIV-1 transactivator (Tat) Protein (47-57).

8. The composition of claim 2, wherein the linker is (succinimidyl 4-(N-maleimidomethyl)cyclohexane-1-carboxylate) (SMCC).

9. The composition of claim 1, wherein the passenger chain oligonucleotide has the nucleotide sequence of SEQ ID NO: 2.

10. The composition of claim 2, wherein the HIV-1 transactivator (Tat) Protein (47-57) further comprises a cysteine residue conjugated to the amino terminus of the peptide SEQ ID NO: 1, and wherein the cysteine residue is further conjugated to a linker, wherein the linker is (succinimidyl 4-(N-maleimidomethyl)cyclohexane-1-carboxylate) (SMCC), and wherein the linker is further conjugated to the passenger chain oligonucleotide, the passenger chain oligonucleotide having an amino group at the 3′ terminus thereof.

11. The composition of claim 10, wherein the composition has the formula I:

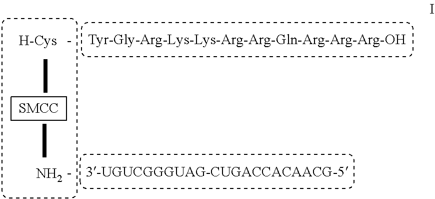

12. The composition of claim 4, wherein the miRNA is miRNA-21 and has a nucleotide sequence of SEQ ID NO: 3.

13. The composition of claim 12, wherein the composition has the formula II:

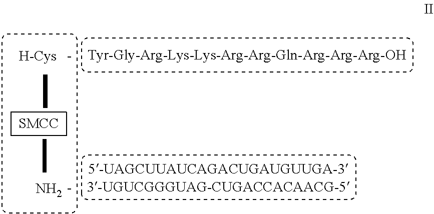

14. A method of delivering an miRNA to a cell, the method comprising contacting a cell with a composition comprising a passenger chain oligonucleotide connected to a cell-penetrating peptide, wherein the cell-penetrating peptide and the passenger chain oligonucleotide are connected by a linker covalently conjugated to the cell-penetrating peptide and to the passenger chain oligonucleotide, and wherein the composition further comprises an miRNA oligonucleotide having a nucleotide sequence selectively hybridized to the passenger chain nucleotide sequence.

15. The method of claim 14, wherein the cell is a cardiac cell.

16. The method of claim 15, wherein the cardiac cell is a cardiomyocyte.

17. The method of claim 14, wherein the composition is administered to a human or animal subject, and wherein the composition is admixed with a pharmaceutically acceptable carrier.

18. The method of claim 17, wherein the pharmaceutically acceptable carrier is a hydrogel generated from decellularized cardiac extracellular matrix.

19. A pharmaceutically acceptable carrier generated from decellularized cardiac extracellular matrix.

20. A therapeutic composition comprising a composition comprising a passenger chain oligonucleotide connected to a cell-penetrating peptide, wherein the cell-penetrating peptide and the passenger chain oligonucleotide are connected by a linker covalently conjugated to the cell-penetrating peptide and to the passenger chain oligonucleotide, and wherein the composition further comprises an miRNA oligonucleotide having a nucleotide sequence selectively hybridized to the passenger chain nucleotide sequence. and a pharmaceutically acceptable carrier.

21. The therapeutic composition of claim 20, wherein the pharmaceutically acceptable carrier is a gel generated from decellularized cardiac extracellular matrix.
